(19) United States
(12) Patent Application Publication
(10) Pub. No.: US 2017/0178052 A1
(43) Pub. Date: Jun. 22, 2017

(54) TECHNOLOGIES FOR STRESS LEVEL MONITORING AND DYNAMIC TASK ASSIGNMENT

(71) Applicants: Lenitra M. Durham, Beaverton, OR (US); Giuseppe Raffa, Portland, OR (US); Glen J. Anderson, Beaverton, OR (US)

(72) Inventors: Lenitra M. Durham, Beaverton, OR (US); Giuseppe Raffa, Portland, OR (US); Glen J. Anderson, Beaverton, OR (US)

(21) Appl. No.: 14/976,517

(22) Filed: Dec. 21, 2015

(51) Int. Cl. G06Q 10/06; G08B 21/18

(52) U.S. Cl. CPC G06Q 10/063116; G08B 21/182

(57) ABSTRACT

Technologies for stress monitoring and task assignment are disclosed. A wearable monitoring system worn by each user of a group of users monitors stress of each user. The wearable monitoring system transmits stress data to a server. The server analyzes the stress data of the users, determines a stress level of each of the users based on the stress data, and assigns one or more tasks to one or more users, based on the determined stress levels.

### BACKGROUND

Mobile computing devices are quickly becoming ubiquitous tools for the average consumer. Mobile computing devices, such as smart phones, smart glasses, tablet computers, and the like, may be used for a variety of purposes including work, entertainment, and information research. As mobile computing devices become more ingrained into the everyday life of users, alternative or additional capabilities are becoming ever more important.

Wearable computing devices, such as smart watches and activity tracking devices, are one type of mobile computing devices that provide a wide range of such additional capabilities. Wearable electronics can provide both input to and output from users to supplement the users' everyday life.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a system for stress monitoring and dynamic task assignment;

FIG. 2 is a simplified block diagram of at least one embodiment of a wearable monitoring device of the system of FIG. 1;

FIG. 3 is a simplified block diagram of at least one embodiment of a server compute device of the system of FIG. 1;

FIG. 4 is a block diagram of at least one embodiment of an environment that may be established by the wearable monitoring device of FIG. 2;

FIG. 5 is a block diagram of at least one embodiment of an environment that may be established by the server compute device of FIG. 4;

FIG. 6 is a simplified flow diagram of at least one embodiment of a method for capturing sensor data that may be executed by a wearable monitoring system of FIG. 1;

FIG. 7 is a simplified flow diagram of at least one embodiment of a method for receiving a task notification that may be executed by the wearable monitoring device of FIG. 1;

FIG. 8 is a simplified block diagram of at least one additional embodiment of a system for stress monitoring and task assignment including a mobile compute device; and

FIGS. 9A and 9B are a simplified block diagram of at least one embodiment of a method for receiving stress data and assigning tasks that may be executed by the server compute device of FIG. 1.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C): (A and B); (B and C); (A and C); or (A, B, and C). Similarly, items listed in the form of “at least one of A, B, or C” can mean (A); (B); (C): (A and B); (B and C); (A or C); or (A, B, and C).

The disclosed embodiments may be implemented, in some cases, in hardware, firmware, software, or any combination thereof. The disclosed embodiments may also be implemented as instructions carried by or stored on one or more transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

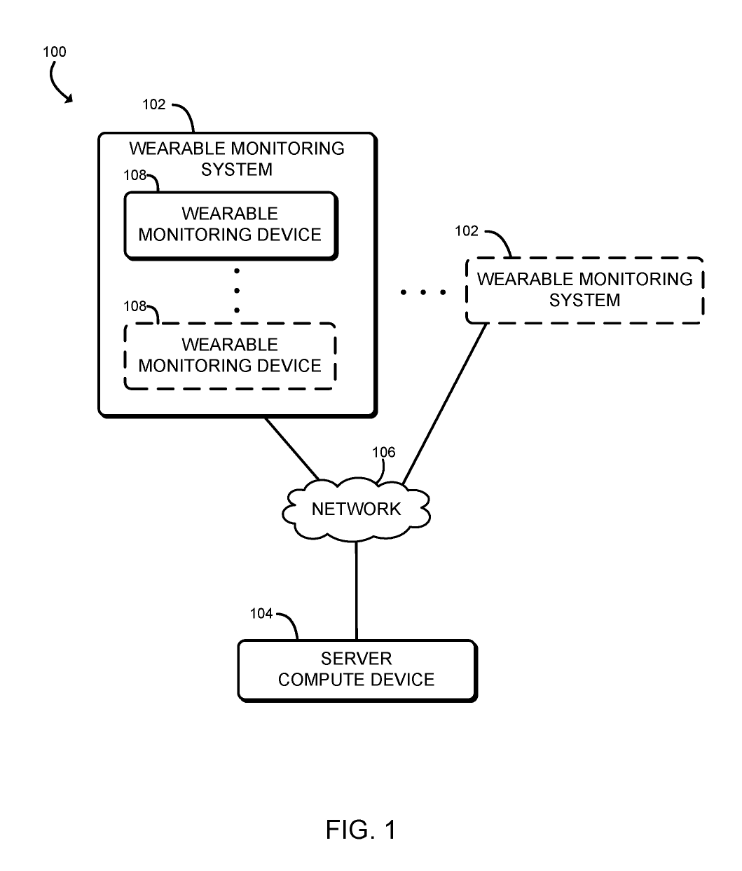

Referring now to FIG. 1, an illustrative system 100 for stress monitoring and dynamic task assignment includes one or more wearable monitoring systems 102 and a server compute device 104, all in communication over a network 106. Each wearable monitoring system 102 includes one or more wearable monitoring devices 108. In use, as discussed in more detail below, each user in a group of users wears a wearable monitoring system 102. The wearable monitoring devices 108 of each wearable monitoring system 102 captures stress data produced by one or more sensors indicative of a characteristic of stress experienced by the corresponding user of that wearable monitoring system 102 while performing a task. For example, a wearable monitoring device 108 may capture data indicative of heart rate, electrodermal activity (EDA), user's voice, etc. The wearable monitoring system 102 aggregates the sensor readings from the wearable monitoring devices 108, and communicates the sensor readings over the network 106 to the server compute device 104.

The server compute device 104 aggregates sensor readings from multiple users (i.e., individuals wearing a wearable monitoring system 102) of the system 100. It should be appreciated that the multiple users do not necessarily need to be wearing all the same types of sensors. For example, one user may be wearing only an EDA sensor, and another user may be wearing a heart rate monitor and breathing monitor (i.e., each wearable monitoring system 102 may include a different number and types of wearable monitoring devices 108). The server compute device 104 determines a level of stress of each user of the system 100, and, in some embodiments, may compare stress across different users of the group, even if they are wearing different types of sensors. The server compute device 104 then assigns tasks to or suggests tasks for the users based on the levels of stress.

In one example setting, the system 100 may be used to monitor the levels of stress of medical residents in a hospital. The medical residents may each wear one or more of the wearable monitoring devices 108 at different times, including both while working and while not working. The system 100 may assign tasks to different residents based on both the tasks that must be completed and the stress level of the residents. Such a system may be preferred to a fixed cap on the number of hours a resident may work, and may provide for increasing the experience a resident receives, while at the same time reducing critical mistakes caused by fatigue and exhaustion (i.e., by a high level of stress). Of course, the system 100 and the technologies disclosed herein are applicable to other industries or situations in which management of user activity is desired.

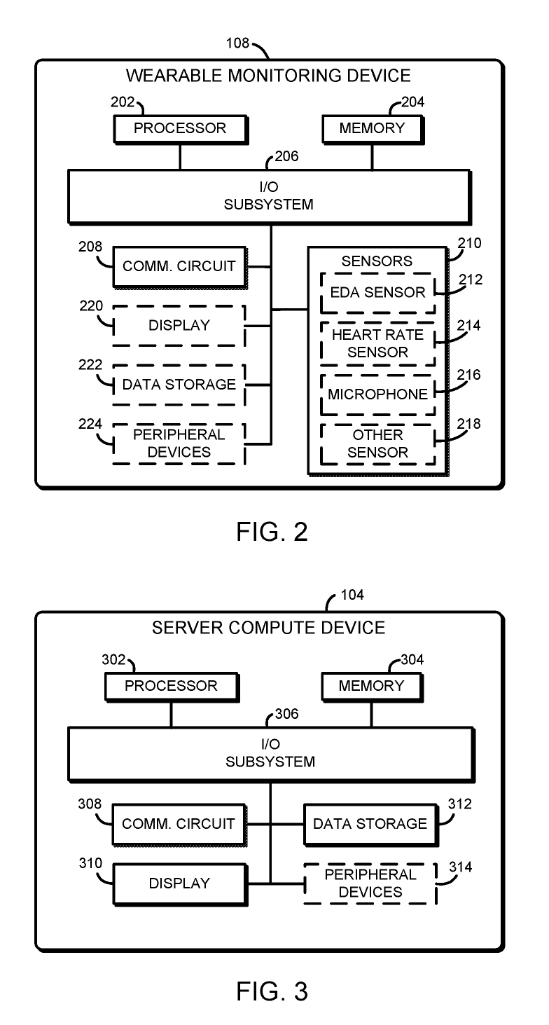

The illustrative wearable monitoring system 102 is embodied as one or more wearable monitoring devices 108. The wearable monitoring devices 108, which are described in more detail below in regards to FIG. 2, communicate with each other, with one wearable monitoring device 108 acting as a “primary” device or controller, with an optional number of additional wearable monitoring devices 108 acting as “secondary” devices or controlled devices. Alternatively, the wearable monitoring devices 108 may communication in a peer-to-peer fashion, or may operate essentially independently with little or no direct communication. Of course, some embodiments may employ a combination of some of all of those approaches. The wearable monitoring devices 108 may communicate with each other using any suitable communication technology and/or protocol including, but not limited to, Ethernet, Bluetooth®, Wi-Fi®, WiMAX, near field communication (NFC), cellular communication, analog communication, serial port communication such as RS-232 or similar direct connection, etc., or some combination thereof.

Each wearable monitoring system 102 and the server compute device 104 are configured to communicate with each other over the network 106. The network 106 may be embodied as any type of communication network capable of facilitating communication between the wearable monitoring system 102 and the server compute device 104. The network 106 may be embodied as or otherwise include Ethernet, Bluetooth®, Wi-Fi®, WiMAX, near field communication (NFC), cellular communication, analog communication, serial port communication such as RS-232 or similar direct connection, etc. In some embodiments, the communication among the wearable monitoring devices 108 may be through the network 106.

Referring now to FIG. 2, each wearable monitoring device 108 may be embodied as any type of wearable device capable of capturing sensor data and performing the functions described herein. For example, an illustrative wearable monitoring device 108 may be embodied as an arm or wrist-worn device such as a smart watch or smart wristband, a head-worn device such as smart glasses, an ear-mounted device such as a headset, clothing with embedded electronics such as a shirt, pair of pants, shoe, labcoat, etc., a head-worn device, a torso-worn device, a belt-mounted device, a device carried in the user's pocket, or any other wearable monitoring device 108. The illustrative embodiment of the wearable monitoring device 108 of FIG. 2 includes a processor 202, a memory 204, an I/O subsystem 206, a communication circuit 208, and sensors 210. In some embodiments, one or more of the illustrative components of the wearable monitoring device 108 may be incorporated in, or otherwise form a portion of, another component. For example, the memory 204, or portions thereof, may be incorporated in the processor 202 in some embodiments. Additionally, in some embodiments, the wearable monitoring device 108 may be implemented in dedicated circuitry to capture sensor data and to transmit the sensor data. Such dedicated circuitry may be embodied as, or otherwise include, analog circuitry, digital circuitry, or some combination thereof, and may not necessarily be able to perform general computing tasks.

The processor 202 may be embodied as any type of processor capable of performing the functions described herein. For example, the processor 202 may be embodied as a single or multi-core processor(s), a single or multi-socket processor, a digital signal processor, a microcontroller, or other processor or processing/controlling circuit. Similarly, the memory 204 may be embodied as any type of volatile or non-volatile memory or data storage capable of performing the functions described herein. In operation, the memory 204 may store various data and software used during operation of the wearable monitoring device 108 such as operating systems, applications, programs, libraries, and drivers. The memory 204 is communicatively coupled to the processor 202 via the I/O subsystem 206, which may be embodied as circuitry and/or components to facilitate input/output operations with the processor 202, the memory 204, and other components of the wearable monitoring device 108. For example, the I/O subsystem 206 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 206 may form a portion of a system-on-a-chip (SoC) and be incorporated, along with the processor 202, the memory 204, and other components of the wearable monitoring device 108, such as the sensors 210, on a single integrated circuit chip.

The communication circuit 208 may be embodied as any type of communication circuit, device, or collection thereof, capable of enabling communications between the wearable monitoring device 108 and other devices such as the server compute device 104 and/or other wearable monitoring devices 108. To do so, the communication circuit 208 may be configured to use any one or more communication technology and associated protocols listed above (e.g., Ethernet, Bluetooth®, Wi-Fi®, WiMAX, NFC, etc.) to effect such communication.

The sensors 210 may be embodied as one or more of any type of sensor capable of producing stress data indicative of a characteristic of stress experienced by the user. For example, the sensors 210 may be embodied as, or otherwise include, an electrodermal activity (EDA) sensor 212, a heart rate sensor 214, a microphone 216, and/or one (or more) other sensor 218. The other sensor 218 may produce any type of stress data indicative of a characteristic of stress experienced by the user, such as a blood pressure monitor, a camera, a skin temperature monitor, a body temperature monitor, an ambient temperature monitor, a sleep monitor, a finger tactile pressure sensing system, a blood flow monitoring system, a movement sensor, a breathing sensor, etc. In some embodiments, the other sensor 218 may include a sensor that may not be directly indicative of a characteristic of stress, such as a Global Positioning System (GPS) circuit, accelerometer, etc. However, even sensors such as those may indicate a characteristic of stress, albeit indirectly (e.g., by generating data from which a stress characteristic may be derived or determined). For example, a GPS circuit may indicate a user is at a certain location, and that location may be associated with a high level of stress (e.g., an operating room). A sensor such as an accelerometer may detect that a user's hand is shaking, which may indicate a high level of stress.

Of course, in other embodiments, the wearable monitoring device 108 may include other or additional components, such as those commonly found in a mobile computing device (e.g., various input/output devices). For example, the wearable monitoring device 108 may also include a display 220, and may have dedicated data storage 222. The optional display 220 may be embodied as any type of display on which information may be displayed to the user of the wearable monitoring device 108, such as a liquid crystal display (LCD), a light emitting diode (LED) display, a cathode ray tube (CRT) display, a plasma display, an image projector (e.g., 2D or 3D), a laser projector, a touchscreen display, a heads-up display, and/or other display technology. The data storage 222 may be embodied as any type of device or devices configured for the short-term or long-term storage of data. For example, the data storage 222 may include any one or more memory devices and circuits, memory cards, hard disk drives, solid-state drives, or other data storage devices.

In some embodiments, the wearable monitoring device 108 may further include one or more peripheral devices 224. Such peripheral devices 224 may include any type of peripheral device commonly found in a wearable device, such as a speaker or headphones, input/output devices, peripheral communication devices, and/or other peripheral devices.

Referring now to FIG. 3, the server compute device 104 may be embodied as any type of sever or similar computing device capable of communicating with the wearable monitoring systems 102 and performing the functions described herein. For example, the server compute device 104 may be embodied as or otherwise be included in, without limitation, a desktop computer, a rack-mounted computer, a smartphone, a cellular phone, a tablet computer, a notebook computer, a laptop computer, a wearable computer, a digital camera, smart eyeglasses, a smart watch, a head-mounted display unit, a handset, a messaging device, a multiprocessor system, a processor-based system, a consumer electronic device, and/or any other computing device capable of performing the function described herein.

The illustrative server compute device 104 includes a processor 302, a memory 304, an I/O subsystem 306, a communication circuit 308, a display 310, data storage 312, and optional peripheral devices 314. Each of the processor 302, the memory 304, the I/O subsystem 306, the communication circuit 308, the display 310, the data storage 312, and the peripheral devices 314 may be similar to the corresponding components of the wearable monitoring device 108. As such, the description of those components of the wearable monitoring device 108 is equally applicable to the description of those components of the server compute device 104 and is not repeated herein for clarity of the description. Of course, the server compute device 104 may include additional peripheral devices 314 not included in the wearable monitoring device 108, such as a hardware keyboard, printing device, etc.

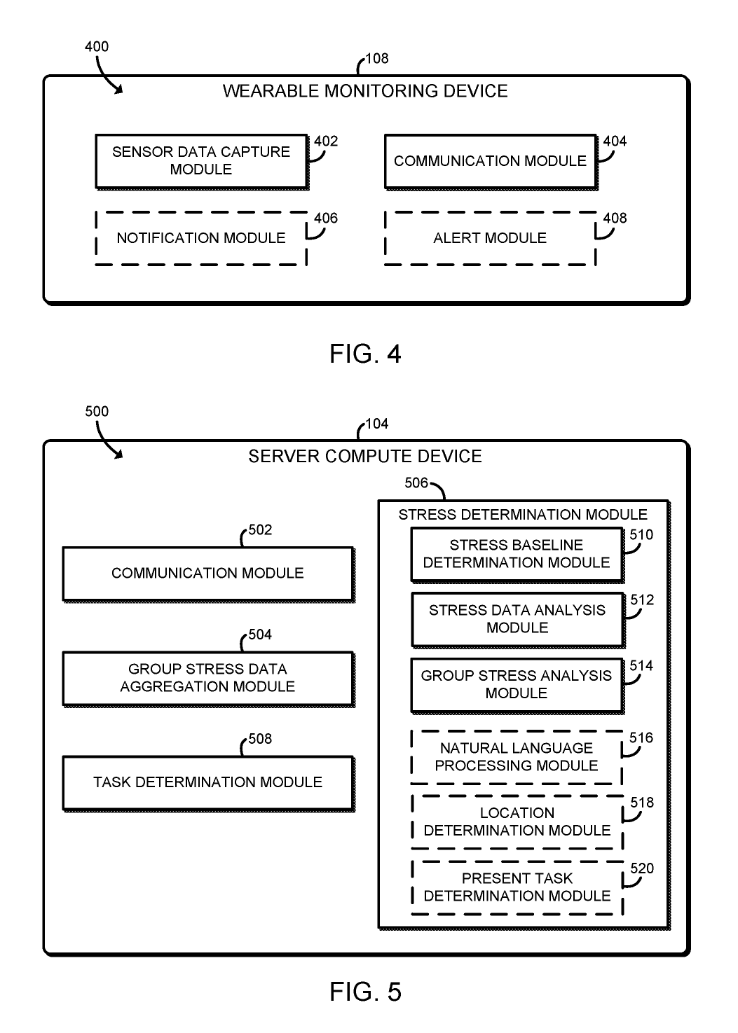

Referring now to FIG. 4, in use, each wearable monitoring device 108 may establish an environment 400. The illustrative environment 400 includes a sensor data capture module 402 and a communication module 404. The various modules of the environment 400 may be embodied as hardware, software, firmware, or a combination thereof. For example, the various modules, logic, and other components of the environment 400 may form a portion of, or otherwise be established by, the processor 202 or other hardware components of the wearable monitoring device 108. As such, in some embodiments, one or more of the modules of the environment 400 may be embodied as circuitry or collection of electrical devices (e.g., a sensor data capture circuit 402, a communication circuit 404, etc.). It should be appreciated that, in such embodiments, one or more of the sensor data capture circuit 402, the communication circuit 404, etc. may form a portion of one or more of the processor 202, the memory 204, the I/O subsystem 206, the data storage 222, the sensors 210, and/or the communication circuit 208. Additionally, in some embodiments, one or more of the illustrative modules may form a portion of another module and/or one or more of the illustrative modules may be independent of one another.

The sensor data capture module 402 is configured to capture or otherwise generate data produced by a sensor. As described above, the sensor may be any type of sensor that is capable of sensing any type of indicator related to stress or characteristic of stress of the user, such as electrodermal activity, heart rate, etc. In some embodiments, the sensor data capture module 402 may be configured to capture sensor data responsively, occasionally, continually, continuously, and/or periodically, e.g., once a second, or may be configured to capture sensor data only when instructed to by the wearable monitoring system 102 and/or the server compute device 104.

The communication module 404 is configured to communicate the captured sensor data to the server compute device 104. In some embodiments, the communication module 404 may communicate data automatically after a data element is captured. In other embodiments, the communication module 404 may only communicate sensor data when requested by the server compute device 104 or transmit a collection of data periodically. Of course, when communication is unavailable (e.g., when out of range of a Wi-Fi® hotspot), the communication module 404 may store data until communication is available, and then transmit the stored data. As discussed above, the communication module 404 may communicate with the server compute device 104 either directly or indirectly through, for example, Ethernet, Bluetooth®, Wi-Fi®, WiMAX, near field communication (NFC), etc. In some embodiments, the communication module 404 is configured to communicate the sensor data to another wearable monitoring device 108, which may then communicate the sensor data to the server compute device 104.

In some embodiments, the wearable monitoring device 108 (e.g., the “primary” wearable monitoring device 108 of the corresponding wearable monitoring system) may also include a notification module 406. The notification module 406 is configured to receive a notification from the server compute device 104. For example, the server compute device 104 may send data related to a new task assigned to or suggested for the user to the wearable monitoring device 108. The notification module 406 receives the new task data, and notifies the user, e.g. by displaying the received task to the user. In some embodiments, the notification module 406 may be configured to update a task or task list by communicating with the server compute device 104, either continually, periodically, and/or in response to a user input.

In some embodiments, the wearable monitoring device 108 may include an alert module 408. The alert module 408 is configured to monitor the stress data and determine if an alert should be generated. For example, the stress data may indicate that the user is operating outside the desired stress levels. Operating outside the desired stress level may mean that one or more stress indicators is a certain amount away from a baseline stress level (discussed in more detail below) for a certain period of time. For example, the alert module 408 may consider the user to be operating outside the desired stress levels if one or more indicators is more than 1, 2, or 3 standard deviations away from the baseline for at least 1, 2, 5, 10 or 15 minutes. In some embodiments, the baseline may be a range. An acceptable range above the baseline or a set of thresholds may also be established in some embodiments. One or more indicators outside the baseline range, outside the acceptable range, or past one or more thresholds at any point in time or for at least 1, 2, 5, 10 or 15 minutes may indicate an undesirable stress level. An indicator may be past a threshold by being above or below the threshold, depending on the threshold. The alert module 408 may be configured to generate an alert in response to such a condition, and may then provide the alert to the user (e.g., by displaying the alert on the display 220). Additionally or alternatively, the alert module 408 may provide the alert to the server compute device 104. The alert module 408 may be configured to provide the alert to the user or not based on the activity of the user. For example, if the user is performing an activity requiring delicate or intense focus such as surgery, the alert module 408 may not provide the alert to the user, in order to prevent distracting him.

Referring now to FIG. 5, in use, the server compute device 104 may establish an environment 500. The illustrative environment 500 includes a communication module 502, a group stress data aggregation module 504, a stress determination module 506, and a task determination module 508. The various modules of the environment 500 may be embodied as hardware, software, firmware, or a combination thereof. For example, the various modules, logic, and other components of the environment 500 may form a portion of, or otherwise be established by, the processor 302 or other hardware components of the server compute device 104. As such, in some embodiments, one or more of the modules of the environment 500 may be embodied as circuitry or collection of electrical devices (e.g., a communication circuit 502, a group stress data aggregation circuit 504, a stress determination circuit 506, etc.). It should be appreciated that, in such embodiments, one or more of the communication circuit 502, group stress data aggregation circuit 504, stress determination circuit 506, etc. may form a portion of one or more of the processor 302, the memory 304, the I/O subsystem 306, the data storage 312, and/or the communication circuit 308. Additionally, in some embodiments, one or more of the illustrative modules may form a portion of another module and/or one or more of the illustrative modules may be independent of one another.

The communication module 502 is configured to communicate with the one or more wearable monitoring devices 108 of one or more wearable monitoring systems 102. For example, in some embodiments, the communication module 502 may communicate with a “primary” wearable monitoring device 108 of each wearable monitoring system 102 of the system 100. The communication module 502 is configured to receive stress data from the one or more wearable monitoring systems 102. In some embodiments, the communication module 502 receives data continuously, continually, or periodically, as described above. In some embodiments, the communications module 502 may request data from a wearable monitoring system 102. As discussed above, the communication module 502 may communicate with the wearable monitoring system 102 either directly or indirectly through, for example, Ethernet, Bluetooth®, Wi-Fi®, WiMAX, near field communication (NFC), etc.

The group stress data aggregation module 504 is configured to aggregate stress data from each wearable monitoring device 108 and/or each wearable monitoring system 102, and associate the stress data with a user of the system 100. The group stress data may be stored using any suitable method, such as in a database.

The stress determination module 506 includes a stress baseline determination module 510, a stress data analysis module 512, and a group stress analysis module 514, and optionally includes a natural language processing module 516, a location determination module 518, and a present task determination module 520. The stress baseline determination module 510 is configured to determine a stress baseline for the user of each wearable monitoring system 102. An initial baseline may be determined by measuring stress levels during non-stressful activities, such as sitting down, reading a book, taking a break, etc. The user may even wear one or more wearable monitoring devices 108 while not working. For example, a smartwatch including several sensors could be worn by a user for several days in order to establish a baseline. In some embodiments, there may be a specific training period used to determine the baseline, as well as a set of acceptable ranges for various stress indicators. In other embodiments, the stress baseline determination module 510 is configured to automatically determine a stress baseline by monitoring the user of the wearable monitoring system 102 over a period of time, such as several hours or several days, as the user is operating in a normal work environment. The stress baseline determination module 510 may analyze that stress data, and determine a baseline based on the varying levels of stress over that time period. In some embodiments, the baseline may be set only during an initial or subsequent training period. In other embodiments, the baseline may be updated continuously, continually, or periodically (e.g., once a day), as new stress data is acquired. Of course, after the initial baseline is determined, the stress baseline determination module 510 may be configured to store the baseline for the user, and then load the baseline for that user at a later time, e.g. at the beginning of every subsequent work shift.

The stress data analysis module 512 is configured to determine a level of stress for one or more users based on the stress data aggregated by the group stress data aggregation module 504. In some embodiments, the stress data analysis module 512 may determine a level of stress for each user for which new data is available. In other embodiments, the stress data analysis module 512 may determine a level of stress only for select users. Similarly, the group stress analysis module 514 is configured to analyze levels of stress across different users from the group of users. For example, the group stress analysis module 514 may sort the users in order from least stress to greatest stress.

In some embodiments, additional modules may be used to analyze, generate, or otherwise provide relevant data for the stress data analysis module 512 to use in determining stress, such as the natural language processing module 516, the location determination module 518, and the present task determination module 520.

The natural language processing module 516 may be configured to analyze the voice data such as word choice, tone of voice, time between responses, etc. of a user and generate a corresponding indicator of stress. The location determination module 518 is configured to determine a location of a user. The location determination module 518 may use data from the user's wearable monitoring system 102 such as data from a GPS sensor. Additionally or alternatively, the location determination module 518 may use signal triangulation or trilateration. Such location determination may be based on cellular signal, Wi-Fi hotspots, etc. In some embodiments, the location may be determined based on data available related to the user, such as location of a current assigned task. The present task determination module 520 is configured to determine a present task of a user. The present task may be determined based on, for example, the location of the user, or on information available related to the user, such as a currently assigned task.

The task determination module 508 is configured to assign tasks based on the determined stress. The tasks available may be generated automatically based on a set of rules, may be manually entered by a manager of the system 100, or some combination thereof. The task determination module 508 may be configured to assign tasks in any number of different ways. For example, the task determination module 508 may determine a set of tasks that are suitable and a set of tasks that are unsuitable for each user, based on that user's stress level. Additionally or alternatively, the task determination module 508 may assign one or more tasks that must be completed to one or more users based on the levels of stress of those users relative to the levels of stress of other members of the group.

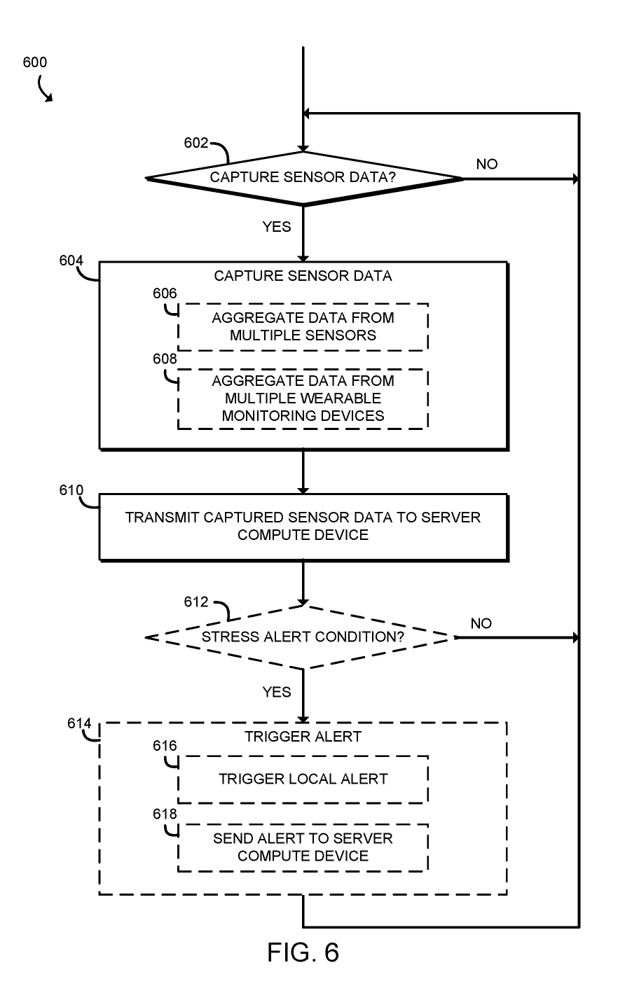

Referring now to FIG. 6, in use, each wearable monitoring system 102 may execute a method 600 for capturing stress data. The method 600 begins with block 602, in which the wearable monitoring system 102 determines whether to capture data. If so, the method 600 proceeds to block 604, and the wearable monitoring system 102 captures sensor data. As described above, in some embodiments, the wearable monitoring system 102 may be configured to continuously, continually, occasionally, or periodically (e.g., once per second) capture stress data produced by at least one sensor. In some embodiments, the wearable monitoring system 102 aggregates data from multiple sensors from one wearable monitoring device 108 in 606. As described above, in embodiments with one “primary” wearable monitoring device 108, the “primary” wearable monitoring device 108 may aggregate data from several “secondary” wearable monitoring devices 108 in block 608. In some embodiments, different sensors 210 and/or different wearable monitoring devices 108 may capture or produce data at different rates. For example, the wearable monitoring system 102 may be configured to capture data from one sensor continually, and may be configured to capture data from another sensor periodically.

In block 610, the wearable monitoring system 102 transmits the captured sensor data to the server compute device 104. As described above, in some embodiments each wearable monitoring device 108 may be configured to transmit the data to the server compute device 104. In other embodiments, each “secondary” wearable monitoring device 108 may be configured to transmit the data to the “primary” wearable monitoring device 108, which then transmits the aggregated data to the server compute device 104.

In embodiments in which a corresponding wearable monitoring device 108 includes an alert module 408, the method 600 may proceed to block 612, in which the corresponding wearable monitoring device 108 of the wearable monitoring system 102 (e.g., the “primary” wearable monitoring device 108) determines if there is a stress alert condition, such as when one or more sensors indicates an undesirable stress level. As described above, this may be determined by comparing each sensor reading with a baseline, a baseline range, an acceptable range, or a threshold.

If the corresponding wearable monitoring device 108 determines there is a stress alert condition, the wearable monitoring device 108 (e.g., the “primary” wearable monitoring device 108) may trigger an alert in block 614. Such alert triggering may include triggering a local alert in block 616, such as by displaying an alert to a user, and/or may include sending an alert to the server compute device 104 in block 618.

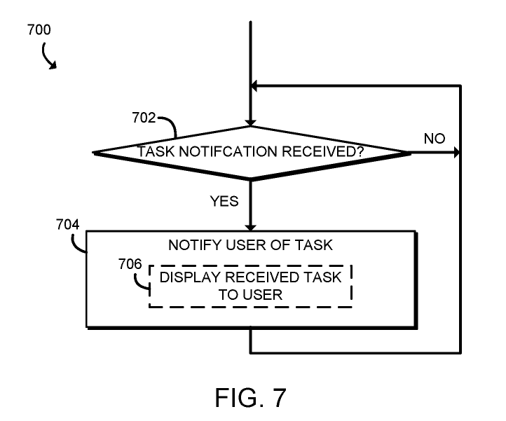

Referring now to FIG. 7, in use, each wearable monitoring system 102 may execute a method 700 for notifying a user in some embodiments. The method 700 begins in block 702, wherein the wearable monitoring system 102 determines if a task notification has been received. If so, the wearable monitoring system 102 notifies the user of the task in block 704. In some embodiments, the wearable monitoring system 102 notifies the user by displaying the received task to the user in block 706.

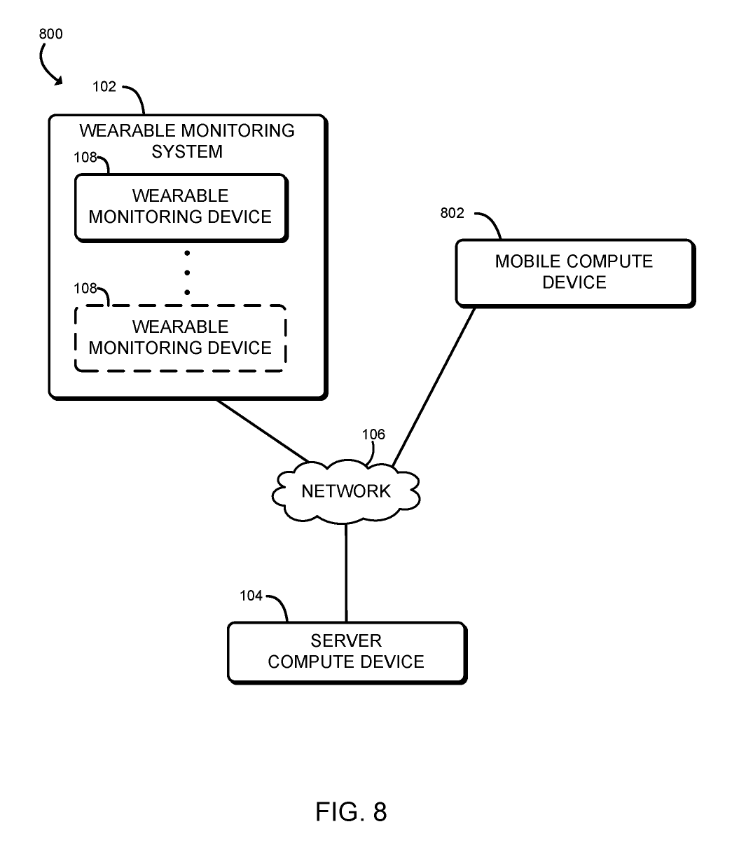

Referring now to FIG. 8, an additional embodiment of a system 800, similar to system 100 of FIG. 1, includes a wearable monitoring system 102 (which includes one or more wearable monitoring devices 108), a server compute device 104, and a network 106. Additionally, the system 800 includes a mobile compute device 802. In the illustrative embodiment of FIG. 8, the mobile compute device 802 may be configured to act in a similar role as the “primary” wearable monitoring device 108 described above. That is, each wearable monitoring device 108 is configured to send sensor data to the mobile compute device 802, which is configured to send the data to the server compute device 104. Of course, the communication between any of the devices of the system 800 may be indirect through the network 106, or may be direct. Additionally or alternatively, the mobile compute device 802 may receive alerts and/or tasks from the wearable monitoring system 102 and/or server compute device 104.

The mobile compute device 802 may be embodied as any type of computing device or system of computing devices capable of performing the functions described herein. For example, in some embodiments, the mobile compute device 802 may be embodied as a smartphone, cellular phone, personal digital assistant, mobile Internet device, wearable computing device, tablet computer, notebook, netbook, desktop computer, laptop computer, server, rack-mounted server, blade server, Ultrabook™, and/or any other computing/communication device. The mobile compute device 802 may include components similar to those of the wearable monitoring device 108 and/or the server compute device 104. The description of those components of the wearable monitoring device 108 and/or the server compute device 104 is equally applicable to the description of components of the mobile compute device 802 and is not repeated herein for clarity of the description. Further, it should be appreciated that the mobile compute device 802 may include other components, sub-components, and devices commonly found in a computing system, which are not discussed above for clarity of the description. In some embodiments, one or more of the components of the wearable monitoring device 108 and/or the server compute device 104 may be omitted from the mobile compute device 802. Of course, the illustrative embodiment of the system 800 may also include additional wearable monitoring systems 102 worn by additional users and which may be associated with corresponding additional mobile compute devices 802, which are not shown for the sake of clarity.

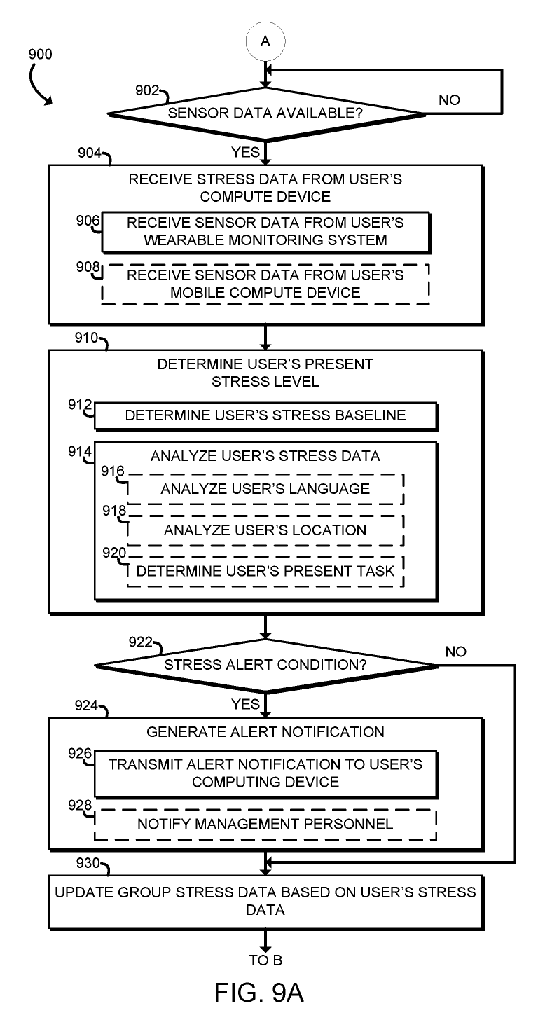

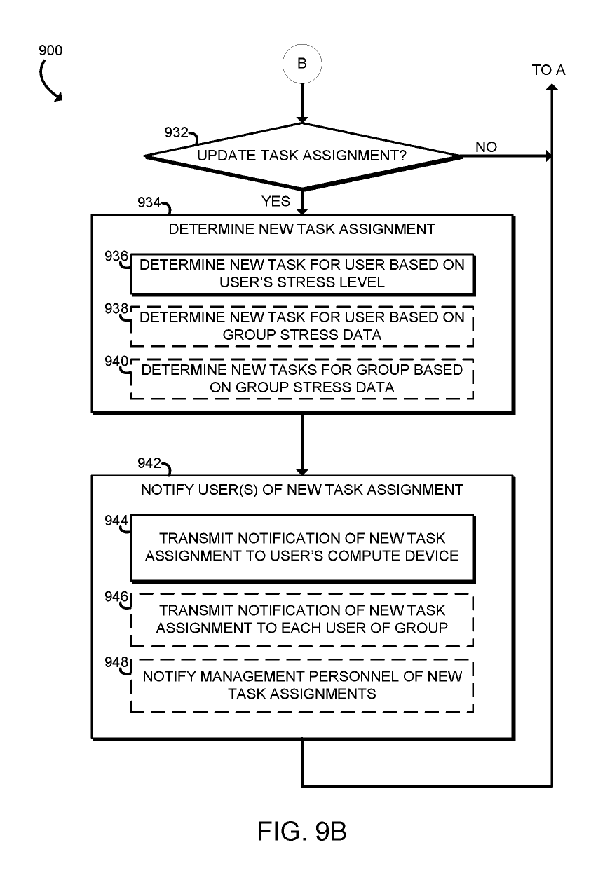

Referring now to FIG. 9A, in use, the server compute device 104 may execute a method 900 for receiving stress data and assigning tasks. The method begins in block 902, wherein the server compute device 104 determines if sensor data is available from one or more wearable monitoring systems 102 of the system 100. If so, the server compute device 104 receives stress data from a user's computing device in block 904. As described above, the stress data may be received in block 906 from each wearable monitoring device 108 of a wearable monitoring system 102 or from the “primary” wearable monitoring device 108 of a wearable monitoring system 102. In some embodiments, the stress data may be received in block 908 from a mobile compute device 802 associated with a user of the wearable monitoring system 102 as discussed above. Of course, in some embodiments, stress data may be received from more than one wearable monitoring system 102, e.g., from multiple wearable monitoring systems 102 associated with different users. The server compute device 104 may aggregate the stress data associated with different users of the group of users.

In block 910, the server compute device 104 determines a user's present level of stress based on the received stress data. In some embodiments, the level of stress may be represented using a single numerical value. In other embodiments, the level of stress may be represented using a more complicated data structure, such as an aggregation of some or all available stress data relating to the user, with or without added data related to analysis of the sensor data.

In order to determine a user's level of stress, the server compute device 104 may determine a particular user's stress baseline in block 912. As described in more detail above, the user's stress baseline may be established during an initial training period and/or may be updated continually or periodically.

In block 914, the server compute device 104 analyzes a user's stress data. The server compute device 104 may employ a wide range of possible analysis techniques. For example, the server compute device 104 may determine a stress level associated with each indicator of stress, and then average all of them. In another example, one or more indicators may be considered outliers, and be discounted from the average. In yet another example, certain indicators may be given a higher weight than others in the average. Of course, analysis techniques other than averaging may be employed as well.

In some embodiments, in block 916, the server compute device 104 may analyze a user's language or voice using natural language processing, as described above. For example, the server compute device 104 may determine the user's voice sounds tired, and that the user is speaking in shorter sentences, or possibly even using incorrect grammar. In block 918, the server compute device 104 may determine and analyze the user's location. For example, if the user in currently in the operating room, that may be taken into account in determining the user's level of stress. In block 920, the server compute device 104 may determine a user's present task. For example, if the user is performing surgery, such a task may be taken into account in analyzing the user's level of stress.

Of course, not all sensors will necessarily provide data at all times. For example, a user may wear different combinations of wearable monitoring devices 108 on different days. The server compute device 104 is capable of determining a level of stress with different combinations of sensor data. In some embodiments, the server compute device 104 may determine a user's performance level of the present task. For example, the finger tactile pressure sensing system may indicate the user is using more or less pressure than normal, e.g. on a scalpel. In some embodiments, the server compute device 104 may determine a user's performance level by receiving an input from a person (e.g., either the user himself, a colleague, a manager, etc.) that is provided to the server compute device 104 (e.g., by entering directly into an input device of the server compute device 104, or by entering input into the wearable monitoring system 102, mobile compute device 802, or other computing device, which then provides the input to the server compute device 104). In some embodiments, the server compute device 104 may generate a report based on the user's stress data, stress history, present performance, and/or past or recent performance.

In block 922, the server compute device 104 determines if there is a stress alert condition based on the analysis of the stress data. A stress alert condition may be determined in a similar fashion as in the wearable monitoring system 102 described above. Of course in some embodiments, different acceptable ranges or thresholds may be used for determining a stress condition in the server compute device 104 as compared to the wearable monitoring system 102. In some embodiments, the acceptable range or thresholds may depend on the present task of the user. If a stress alert condition exists, an alert notification may be generated in block 924. This alert notification may be transmitted to the user's computing device (e.g., to a wearable monitoring device 108 worn by the user, to the user's mobile compute device 802, etc.) in block 926. In some embodiments, the alert notification may be additionally or alternatively provided to management personnel in block 928.

After generating the alert or determining there is no stress alert condition, the server compute device 104 updates the group stress data based on the determined user's stress data in block 930. In some embodiments, this may include updating a database or similar data structure with the updated stress data. Additionally, this may include sorting the users of the group based on the updated stress data and the determined stress levels.

The method 900 then proceeds to block 932 in FIG. 9B, wherein server compute device 104 determines if task assignments should be updated. In some embodiments, task assignments may be updated periodically, e.g. once an hour or once a shift. Additionally or alternatively, task assignments may be updated in response to certain stress levels. For example, a user may be reassigned to a new task if the user's stress level is determined to be too high for the user's present task. In some embodiments, the task assignments may be updated in response to input from a user, such as a member of management.

In block 934, the server compute device 104 may determine a new task assignment for one or more users. To do so, in block 936, the server compute device 104 may determine a new task for a user based on the user's present stress level as determined in block 910. Additionally or alternatively, in block 938, the server compute device 104 may determine a new task for a user based on group stress data. For example, the levels of stress of users of the group may be sorted, and a user may be given a task based on the user's level of stress compared to other users of the group. Of course, the server compute device 104 may also determine new tasks for several or all members of the group based on the group stress data in block 940. In some cases, the server compute device 104 may assign a user to take a break. In some embodiments, assigning a task includes suggesting a task (i.e., the assigning may be tentative or suggestive in nature, and not a final determination that a user is required to perform a task).

The server compute device 104 may take into account many different factors in determining assigned tasks. For example, the server compute device 104 may take into account the length of time of a task, the difficulty of the task, the training required, the experience of the user, past performance of the user under similar stress conditions as the present, performance of similar tasks in recent history, etc. In some embodiments, the server compute device 104 may take into account dynamic factors such as if two users work well together or not (i.e., if two users are already stressed, and their stress levels increase when they work together, do not assign them to the same task). The server compute device 104 may determine a both a list of tasks that are suitable for a user and a list of tasks that are unsuitable for the user, based on the user's stress level.

In block 942, the server compute device 104 notifies one or more users of the new task assignment. In the illustrative embodiment, a notification is transmitted to the user's computing device (e.g., to a wearable monitoring device 108 worn by the user, to the user's mobile compute device 802, etc.) in block 944. Of course, in block 946, a notification may be sent to several users of the group if one or more new tasks were assigned to several users of the group. In some embodiments, management personnel may be additionally or alternatively notified of the new assignments in block 948. Further, the management personnel may be able to approve, modify, or cancel certain assignments, and may then be able to send out the notification to users.

### EXAMPLES

Illustrative examples of the devices, systems, and methods disclosed herein are provided below. An embodiment of the devices, systems, and methods may include any one or more, and any combination of, the examples described below.

Example 1 includes a server compute device for monitoring stress level of a user, the server compute device comprising a communication module to receive, from a compute device of the user, stress data indicative of a characteristic of stress experienced by the user while performing a task; a stress determination module to determine, based on the stress data, a level of stress of the user; and a task determination module to determine, based on the level of stress of the user, a new task to be assigned to the user.

Example 2 includes the subject matter of Example 1, and wherein the communication module is further to notify the user of the new task.

Example 3 includes the subject matter of any of Examples 1 and 2, and wherein to determine the new task to be assigned to the user comprises to determine the new task to be assigned to the user further based on one or more of a length of the new task, a difficulty of the new task, an amount of training required for the new task, an amount of experience of the user required for the new task, a past performance of the user on the new task, or a recent performance of the user.

Example 4 includes the subject matter of any of Examples 1-3, and wherein the communication module is further to receive, from one or more additional compute devices of one or more additional users, additional stress data indicative of one or more additional characteristics of stress experienced by the one or more additional users while performing one or more additional tasks; and the stress determination module is further to determine, based on the additional stress data, a level of stress of the one or more additional users, wherein to determine the new task to be assigned to the user comprises to determine the new task to be assigned to the user further based on the level of stress of the one or more additional users.

Example 5 includes the subject matter of any of Examples 1-4, and wherein the stress data is generated by a first set of sensors, each sensor of the first set of sensors having a type, the types of the first set of sensors defining a first set of types of sensors; the additional stress data is generated by a second set of sensors, each sensor of the second set of sensors having a type, the types of the second set of sensors defining a second set of types of sensors; and the first set of types of sensors is different from the second set of types of sensors.

Example 6 includes the subject matter of any of Examples 1-5, and wherein the new task comprises taking a break from the task.

Example 7 includes the subject matter of any of Examples 1-6, and wherein the stress determination module is further to obtain a performance level of the user on the task, and generate a report based on the performance level and the level of stress.

Example 8 includes the subject matter of any of Examples 1-7, and wherein the stress determination module is further to generate an alert based on a determination that the level of stress is outside an acceptable range.

Example 9 includes the subject matter of any of Examples 1-8, and wherein the communication module is to send the alert to the compute device of the user.

Example 10 includes the subject matter of any of Examples 1-9, and wherein the stress determination module is further to determine a present activity of the user, and determine whether to send the alert based on the present activity of the user.

Example 11 includes the subject matter of any of Examples 1-10, and wherein the stress determination module is further to provide the alert to management personnel associated with the user.

Example 12 includes the subject matter of any of Examples 1-11, and wherein the acceptable range is dependent on the task of the user.

Example 13 includes the subject matter of any of Examples 1-12, and wherein the stress data includes voice data of the user, and wherein to determine the level of stress of the user comprises to determine, further based on natural language processing of the voice data, the level of stress of the user.

Example 14 includes the subject matter of any of Examples 1-13, and wherein the task determination module is further to determine one or more unsuitable tasks for the user based on the level of stress of the user.

Example 15 includes a compute device for monitoring stress of a user of the compute device, the compute device comprising a communication module to obtain stress data produced by at least one sensor while the user performs a task, wherein the stress data is indicative of a characteristic of stress experienced by the user while performing the task; transmit the stress data to a server; and receive a new task to be performed by the user, wherein the new task is determined by the server based on the stress data.

Example 16 includes the subject matter of Example 15, and wherein the compute device comprises a mobile compute device, and wherein to obtain the stress data produced by the at least one sensor comprises to receive, from a wearable monitoring device of the user, the stress data produced by at least one sensor of the wearable monitoring device.

Example 17 includes the subject matter of any of Examples 15 and 16, and wherein the compute device comprises a wearable monitoring device, the wearable monitoring device comprising the at least one sensor; and a sensor data capture module to capture the stress data, wherein to obtain the stress data produced by the at least one sensor comprises to obtain the stress data from the sensor data capture module.

Example 18 includes the subject matter of any of Examples 15-17, and wherein the new task comprises taking a break from the task.

Example 19 includes the subject matter of any of Examples 15-18, and further including an alert module to generate an alert based on a determination that the level of stress is outside an acceptable range.

Example 20 includes the subject matter of any of Examples 15-19, and further including a display, and wherein the alert module is further to display the alert on a display of the compute device.

Example 21 includes the subject matter of any of Examples 15-20, and wherein the communication module is further to provide the alert to management personnel.

Example 22 includes the subject matter of any of Examples 15-21, and wherein the acceptable range is dependent on the task of the user.

Example 23 includes a method for monitoring stress level of a user, the method comprising receiving, by a server and from a compute device of the user, stress data indicative of a characteristic of stress experienced by the user while performing a task; determining, by the server and based on the stress data, a level of stress of the user; and determining, by the server and based on the level of stress of the user, a new task to be assigned to the user.

Example 24 includes the subject matter of Example 23, and further including notifying, by the server, the user of the new task.

Example 25 includes the subject matter of any of Examples 23 and 24, and wherein determining the new task to be assigned to the user comprises determining the new task to be assigned to the user further based on one or more of a length of the new task, a difficulty of the new task, an amount of training required for the new task, an amount of experience of the user required for the new task, a past performance of the user on the new task, or a recent performance of the user.

Example 26 includes the subject matter of any of Examples 23-25, and further including receiving, by the server and from one or more additional compute devices of one or more additional users, additional stress data indicative of one or more additional characteristics of stress experienced by the one or more additional users while performing one or more additional tasks; and determining, by the server and based on the additional stress data, a level of stress of the one or more additional users, wherein determining the new task to be assigned to the user comprises determining the new task to be assigned to the user further based on the level of stress of the one or more additional users.

Example 27 includes the subject matter of any of Examples 23-26, and wherein the stress data is generated by a first set of sensors, each sensor of the first set of sensors having a type, the types of the first set of sensors composing a first set of types of sensors; the additional stress data is generated by a second set of sensors, each sensor of the second set of sensors having a type, the types of the second set of sensors composing a second set of types of sensors; and the first set of types of sensors is different from the second set of types of sensors.

Example 28 includes the subject matter of any of Examples 23-27, and wherein the new task comprises taking a break from the task.

Example 29 includes the subject matter of any of Examples 23-28, and further including obtaining, by the server compute device, a performance level of the user on the task; and generating a report based on the performance level and the level of stress.

Example 30 includes the subject matter of any of Examples 23-29, and further including determining, by the server compute device, whether the level of stress is outside an acceptable range; and generating, by the server compute device, an alert based on a determination that the level of stress is outside the acceptable range.

Example 31 includes the subject matter of any of Examples 23-30, and further including sending the alert to the compute device of the user.

Example 32 includes the subject matter of any of Examples 23-31, and further including determining, by the server compute device, a present activity of the user, and determining, by the server compute device and prior to sending the alert, whether to send the alert based on the present activity of the user.

Example 33 includes the subject matter of any of Examples 23-32, and further including providing the alert to management personnel.

Example 34 includes the subject matter of any of Examples 23-33, and wherein the acceptable range depends on the task of the user.

Example 35 includes the subject matter of any of Examples 23-34, and wherein the stress data includes voice data of the user, and wherein determining the level of stress of the user comprises determining, further based on natural language processing of the voice data, the level of stress of the user.

Example 36 includes the subject matter of any of Examples 23-35, and further including determining, by the server compute device, one or more unsuitable tasks for the user based on the level of stress of the user.

Example 37 includes a method for monitoring stress of a user of a compute device, the method comprising obtaining, by the compute device, stress data produced by at least one sensor while the user performs a task, wherein the stress data is indicative of a characteristic of stress experienced by the user while performing the task; transmitting, by the compute device, the stress data to a server; and receiving, by the compute device, a new task to be performed by the user and determined by the server based on the stress data.

Example 38 includes the subject matter of Example 37, and wherein the compute device comprises a mobile compute device, and wherein obtaining the stress data produced by at least one sensor comprises receiving, from a wearable monitoring device of the user, the stress data produced by at least one sensor of the wearable monitoring device.

Example 39 includes the subject matter of any of Examples 37 and 38, and wherein the compute device comprises a wearable monitoring device, and wherein obtaining the stress data produced by at least one sensor comprises capturing the stress data produced by at least one sensor of the wearable monitoring device.

Example 40 includes the subject matter of any of Examples 37-39, and wherein the new task comprises taking a break from the task.

Example 41 includes the subject matter of any of Examples 37-40, and further including generating, by the compute device, an alert based on the level of stress being outside an acceptable range.

Example 42 includes the subject matter of any of Examples 37-41, and further including displaying the alert on a display of the compute device.

Example 43 includes the subject matter of any of Examples 37-42, and further including providing the alert to management personnel.

Example 44 includes the subject matter of any of Examples 37-43, and wherein the acceptable range depends on the task of the user.

Example 45 includes one or more machine-readable storage media comprising a plurality of instructions stored thereon that in response to being executed result in a compute device performing the compute device of any of Examples 23-44.

Example 46 includes a server compute device for monitoring stress level of a user, the server compute device comprising means for receiving, from a compute device of the user, stress data indicative of a characteristic of stress experienced by the user while performing a task; means for determining, based on the stress data, a level of stress of the user; and means for determining, based on the level of stress of the user, a new task to be assigned to the user.

Example 47 includes the subject matter of Example 46, and further including means for notifying the user of the new task.

Example 48 includes the subject matter of any of Examples 46 and 47, and wherein the means determining the new task to be assigned to the user comprises means for determining the new task to be assigned to the user further based on one or more of a length of the new task, a difficulty of the new task, an amount of training required for the new task, an amount of experience of the user required for the new task, a past performance of the user on the new task, or a recent performance of the user.

Example 49 includes the subject matter of any of Examples 46-48, and further including means for receiving, from one or more additional compute devices of one or more additional users, additional stress data indicative of one or more additional characteristics of stress experienced by the one or more additional users while performing one or more additional tasks; and means for determining, based on the additional stress data, a level of stress of the one or more additional users, wherein the means for determining the new task to be assigned to the user comprises means for determining the new task to be assigned to the user further based on the level of stress of the one or more additional users.

Example 50 includes the subject matter of any of Examples 46-49, and wherein the stress data is generated by a first set of sensors, each sensor of the first set of sensors having a type, the types of the first set of sensors composing a first set of types of sensors; the additional stress data is generated by a second set of sensors, each sensor of the second set of sensors having a type, the types of the second set of sensors composing a second set of types of sensors; and the first set of types of sensors is different from the second set of types of sensors.

Example 51 includes the subject matter of any of Examples 46-50, and wherein the new task comprises taking a break from the task.

Example 52 includes the subject matter of any of Examples 46-51, and further including means for obtaining a performance level of the user on the task; and means for generating a report based on the performance level and the level of stress.

Example 53 includes the subject matter of any of Examples 46-52, and, further including means for determining whether the level of stress is outside an acceptable range; and means for generating an alert based on a determination that the level of stress is outside the acceptable range.

Example 54 includes the subject matter of any of Examples 46-53, and further including means for sending the alert to the compute device of the user.

Example 55 includes the subject matter of any of Examples 46-54, and further including means for determining a present activity of the user, and means for determining, prior to sending the alert, whether to send the alert based on the present activity of the user.

Example 56 includes the subject matter of any of Examples 46-55, and further including means for providing the alert to management personnel.

Example 57 includes the subject matter of any of Examples 46-56, and wherein the acceptable range depends on the task of the user.

Example 58 includes the subject matter of any of Examples 46-57, and wherein the stress data includes voice data of the user, and wherein the means for determining the level of stress of the user comprises means for determining, further based on natural language processing of the voice data, the level of stress of the user.

Example 59 includes the subject matter of any of Examples 46-58, and further including means for determining one or more unsuitable tasks for the user based on the level of stress of the user.

Example 60 includes a compute device for monitoring stress of a user of the compute device, the compute device comprising means for obtaining stress data produced by at least one sensor while the user performs a task, wherein the stress data is indicative of a characteristic of stress experienced by the user while performing the task; means for transmitting the stress data to a server; and means for receiving a new task to be performed by the user and determined by the server based on the stress data.

Example 61 includes the subject matter of Example 60, and wherein the compute device comprises a mobile compute device, and wherein the means for obtaining the stress data produced by at least one sensor comprises means for receiving, from a wearable monitoring device of the user, the stress data produced by at least one sensor of the wearable monitoring device.

Example 62 includes the subject matter of any of Examples 60 and 61, and wherein the compute device comprises a wearable monitoring device, and wherein means for obtaining the stress data produced by at least one sensor comprises means for capturing the stress data produced by at least one sensor of the wearable monitoring device.

Example 63 includes the subject matter of any of Examples 60-62, and wherein the new task comprises taking a break from the task.

Example 64 includes the subject matter of any of Examples 60-63, and further including means for generating, by the compute device, an alert based on the level of stress being outside an acceptable range.

Example 65 includes the subject matter of any of Examples 60-64, and further including means for displaying the alert on a display of the compute device.

Example 66 includes the subject matter of any of Examples 60-65, and further including means for providing the alert to management personnel.

Example 67 includes the subject matter of any of Examples 60-66, and wherein the acceptable range depends on the task of the user.

1. A server compute device comprising:
a communication module to receive, from a compute device of a user, stress data indicative of a characteristic of stress experienced by the user while performing a task;
a stress determination module to determine, based on the stress data, a level of stress of the user; and
a task determination module to determine, based on the level of stress of the user, a new task to be assigned to the user.

2. The server compute device of claim 1, wherein the communication module is further to notify the user of the new task.

3. The server compute device of claim 1, wherein to determine the new task to be assigned to the user comprises to determine the new task to be assigned to the user further based on one or more of a length of the new task, a difficulty of the new task, an amount of training required for the new task, an amount of experience of the user required for the new task, a past performance of the user on the new task, or a recent performance of the user.

4. The server compute device of claim 1, wherein:
the communication module is further to receive, from one or more additional compute devices of one or more additional users, additional stress data indicative of one or more additional characteristics of stress experienced by the one or more additional users while performing one or more additional tasks; and
the stress determination module is further to determine, based on the additional stress data, a level of stress of the one or more additional users,
wherein to determine the new task to be assigned to the user comprises to determine the new task to be assigned to the user further based on the level of stress of the one or more additional users.

5. The server compute device of claim 4, wherein:
the stress data is generated by a first set of sensors, each sensor of the first set of sensors having a type, the types of the first set of sensors defining a first set of types of sensors;
the additional stress data is generated by a second set of sensors, each sensor of the second set of sensors having a type, the types of the second set of sensors defining a second set of types of sensors; and
the first set of types of sensors is different from the second set of types of sensors.

6. The server compute device of claim 1, wherein the stress determination module is further to:
obtain a performance level of the user on the task, and
generate a report based on the performance level and the level of stress.

7. The server compute device of claim 1, wherein the stress determination module is further to generate an alert based on a determination that the level of stress is outside an acceptable range.

8. The server compute device of claim 7, wherein the stress determination module is further to:
determine a present activity of the user, and
determine whether to send the alert to the compute device of the user based on the present activity of the user.

9. A compute device for monitoring stress of a user of the compute device, the compute device comprising a communication module to:
obtain stress data produced by at least one sensor while the user performs a task, wherein the stress data is indicative of a characteristic of stress experienced by the user while performing the task;
transmit the stress data to a server; and
receive a new task to be performed by the user, wherein the new task is determined by the server based on the stress data.

10. The compute device of claim 9, wherein the compute device comprises a wearable monitoring device, the wearable monitoring device comprising:
the at least one sensor; and
a sensor data capture module to capture the stress data,
wherein to obtain the stress data produced by the at least one sensor comprises to obtain the stress data from the sensor data capture module.

11. The compute device of claim 9, further comprising an alert module to generate an alert based on a determination that the level of stress is outside an acceptable range.

12. The compute device of claim 11, wherein the acceptable range is dependent on the task of the user.

13. One or more machine-readable storage media comprising a plurality of instructions stored thereon that, when executed, cause a server compute device to:
receive, from a compute device of the user, stress data indicative of a characteristic of stress experienced by the user while performing a task;
determine, based on the stress data, a level of stress of the user; and
determine, based on the level of stress of the user, a new task to be assigned to the user.

14. The one or more machine-readable media of claim 13, wherein the plurality of instructions further cause the server compute device to notify the user of the new task.

15. The one or more machine-readable media of claim 13, wherein to determine the new task to be assigned to the user comprises to determine the new task to be assigned to the user further based on one or more of a length of the new task, a difficulty of the new task, an amount of training required for the new task, an amount of experience of the user required for the new task, a past performance of the user on the new task, or a recent performance of the user.

16. The one or more machine-readable media of claim 13, wherein the plurality of instructions further cause the server compute device to:
receive, from one or more additional compute devices of one or more additional users, additional stress data indicative of one or more additional characteristics of stress experienced by the one or more additional users while performing one or more additional tasks; and
determine, based on the additional stress data, a level of stress of the one or more additional users,
wherein to determine the new task to be assigned to the user comprises to determine the new task to be assigned to the user further based on the level of stress of the one or more additional users.

17. The one or more machine-readable media of claim 16, wherein:
the stress data is generated by a first set of sensors, each sensor of the first set of sensors having a type, the types of the first set of sensors defining a first set of types of sensors;
the additional stress data is generated by a second set of sensors, each sensor of the second set of sensors having a type, the types of the second set of sensors defining a second set of types of sensors; and
the first set of types of sensors is different from the second set of types of sensors.

18. The one or more machine-readable media of claim 13, wherein the plurality of instructions further cause the server compute device to:
obtain a performance level of the user on the task, and
generate a report based on the performance level and the level of stress.

19. The one or more machine-readable media of claim 13, wherein the plurality of instructions further cause the server compute device to generate an alert based on a determination that the level of stress is outside an acceptable range.

20. The one or more machine-readable media of claim 19, wherein the plurality of instructions further cause the server compute device to:
determine a present activity of the user, and
determine whether to send the alert to the compute device of the user based on the present activity of the user.

21. A method comprising:
receiving, by a server and from a compute device of a user, stress data indicative of a characteristic of stress experienced by the user while performing a task;
determining, by the server and based on the stress data, a level of stress of the user; and
determining, by the server and based on the level of stress of the user, a new task to be assigned to the user.

22. The method of claim 21, further comprising:
receiving, by the server and from one or more additional compute devices of one or more additional users, additional stress data indicative of one or more additional characteristics of stress experienced by the one or more additional users while performing one or more additional tasks; and
determining, by the server and based on the additional stress data, a level of stress of the one or more additional users,
wherein determining the new task to be assigned to the user comprises determining the new task to be assigned to the user further based on the level of stress of the one or more additional users.

23. The method of claim 22, wherein:
the stress data is generated by a first set of sensors, each sensor of the first set of sensors having a type, the types of the first set of sensors composing a first set of types of sensors;
the additional stress data is generated by a second set of sensors, each sensor of the second set of sensors having a type, the types of the second set of sensors composing a second set of types of sensors; and
the first set of types of sensors is different from the second set of types of sensors.

24. The method of claim 21, further comprising:
determining, by the server compute device, whether the level of stress is outside an acceptable range; and
generating, by the server compute device, an alert based on a determination that the level of stress is outside the acceptable range.

25. The method of claim 24, further comprising:
determining, by the server compute device, a present activity of the user, and
determining, by the server compute device and prior to sending the alert, whether to send the alert to the compute device of the user based on the present activity of the user.
